SEQUENCE LISTING (3 sequences)

SEQ ID NO: 1 - DNA, 24 bp, Artificial Sequence
  Synthetic oligonucleotide
ctaacaggcgcatacgatctgaca

SEQ ID NO: 2 - DNA, 24 bp, Artificial Sequence
  Synthetic oligonucleotide
tacgcatagcgatctccttcgttg

SEQ ID NO: 3 - DNA, 26 bp, Artificial Sequence
  Synthetic oligonucleotide
aatatgctgcctactgccctacgctt

(19) United States
(12) Patent Application Publication
(10) Pub. No.: US 2009/0181441 A1
(43) Pub. Date: Jul. 16, 2009

(54) Porous silicon-polymer composites for biosensor applications

(75) Inventors: Joon-Hyung Jin, Seoul (KR); Evangelyn C. Alocilja, East Lansing, MI (US); Daniel L. Grooms, Williamston, MI (US)

Correspondence Address: Ian C. McLeod;IAN C. McLEOD, P.C., 2190 Commons Parkway, Okemos, MI, 48864, US

(73) Assignee: Board of Trustees of Michigan State University, East Lansing, MI (US)

(21) Appl. No.: 12/313,996

(22) Filed: Nov. 26, 2008

Related U.S. Application Data
(60) Provisional application No. 61/004,343, filed on Nov. 27, 2007.

(51) Int. Cl. C12N 11/08; G01N 30/96; C23C 16/00; B01L 3/00

(52) U.S. Cl. 435/180; 422/69; 205/196; 422/61

(57) ABSTRACT

The disclosure relates to porous silicon/conductive polymer composites that can be used in biosensor applications (e.g., in a binding assay that captures a target analyte). The composite material generally includes (a) a p-doped silicon substrate that has a porous surface; (b) a lawn of a conductive polymer bound to the porous surface; and (c) a binding pair member bound to the conductive polymer. The porous silicon surface provides excellent adhesion between the substrate and the conductive polymer, thereby eliminating the need for an intervening metallic layer. Processes according to the disclosure for forming the composite material generally include electropolymerizing and electrodepositing the conductive polymer onto the porous surface of the silicon substrate and then binding the binding pair member to the conductive polymer. Methods and kits employing the composite materials are also disclosed.

### CROSS REFERENCE TO RELATED APPLICATIONS

Priority to U.S. Provisional Application Ser. No. 61/004,343, filed Nov. 27, 2007, which is incorporated herein by reference in its entirety, is claimed.

### REFERENCE TO A COMPUTER LISTING APPENDIX SUBMITTED ON A COMPACT DISC

The application contains nucleotide sequences which are identified with SEQ ID NOs. A compact disc is provided which contains the Sequence Listings for the sequences. The Sequence Listing on the compact disc is identical to the paper copy of the Sequence Listing provided with the application.

### BACKGROUND OF THE DISCLOSURE

1. Field of the Disclosure

The present disclosure relates to an assay which uses porous p-doped silicon (e.g., boron-doped) as a substrate for a lawn of a conductive polymer (e.g., polypyrrole doped with a perchlorate anion electrolyte) that is labeled with a member of a binding pair. The porous silicon substrate enables a denser and more reliable lawn of electropolymerized conductive polymer to be bound to the silicon substrate for the assay. Any type of assay which uses a bound member of a binding pair can use the composite material of the present disclosure.

2. Brief Description of Related Technology

Conductive polymers (CPs) are used in chemical and biological sensors as an immobilizing matrix for probe materials, such as enzymes, DNAs and antibodies. Conductive polymers have good compatibility with biomaterials. If conductive polymers could be combined with silicon substrate, then mass production and miniaturization of a sensing device could be possible; however, direct immobilization of a conductive polymer on a silicon substrate is difficult because the surface of an unmodified silicon wafer is too smooth and too chemically hydrophobic to permit effective immobilization of the conductive polymer. Therefore, a metallic layer is commonly deposited on the silicon substrate as an intermediate layer between the substrate and the conductive polymer when fabricating silicon-based sensors with conductive polymer lawns.

Polypyrroles have been used as conductors on modified substrates (e.g., metal-coated silicon) for conductimetric assays, for example as indicated by Hamers et al. U.S. Publication No. 2006/0014155. Other publications describing polypyrroles for use in assays include Briones et al. U.S. Publication No. 2003/0113229, Ward et al. U.S. Publication No. 2004/0018611, and International Publication No. WO 00/77523.

### OBJECTS

There is a need for electropolymerization of conducting polymers directly on the silicon substrate without any deposition of a metallic layer. It is therefore an object of the present disclosure to provide an improved silicon substrate bound to a conducting polymer for use in a conductimetric assay as a biosensor. It is further an object to provide a reliable and economic assay.

These and other objects may become increasing apparent by reference to the following description and drawings.

### SUMMARY

The disclosure relates to porous silicon/conductive polymer composite materials that can be used in biosensor applications when an analyte-specific binding pair member is bound to the conductive polymer. Processes according to the disclosure for forming the composite material generally include electropolymerizing and electrodepositing the conductive polymer onto the porous surface of the silicon substrate and then binding the binding pair member to the conductive polymer. Methods and kits employing the composite materials are also disclosed.

In one embodiment, a composite material for use in a binding assay that captures an analyte is disclosed. The composite material comprises: (a) a p-doped silicon substrate comprising a porous surface; (b) a lawn of a conductive polymer bound to the porous surface; and (c) a binding pair member bound to the conductive polymer. In a more specific embodiment, the composite material comprises: (a) a p-doped silicon substrate comprising a porous surface and a boron dopant; (b) a lawn of a conductive polymer bound to the porous surface, the conductive polymer comprising one or more of an electrolyte-doped polypyrrole, polyaniline, and polythiophene; and (c) a probe DNA (pDNA) oligonucleotide electrostatically bound to the conductive polymer.

In another embodiment, a process for forming the composite material comprises: (a) providing a p-doped silicon substrate comprising a porous surface; (b) electrodepositing a lawn of a conductive polymer onto to the porous surface; and (c) binding a binding pair member to the conductive polymer, thereby forming the composite material. In a refinement, part (a) of the process further comprises (a-1) providing a p-doped crystalline silicon substrate, (a-2) etching pores into a surface of the p-doped crystalline silicon substrate (e.g., by using an acid etchant solution and by applying an anodizing current), thereby forming a porous silicon layer in the p-doped crystalline silicon substrate, the porous silicon layer comprising the porous surface; and, optionally, (a-3) annealing the porous silicon layer, for example after a silicon oxide layer has formed on the porous surface. Electrodeposition of the conductive polymer lawn preferably comprises electropolymerizing one or more conductive monomers by at least one of cyclic voltammetry, chronoamperometry, and chronopotentiometry. Electrodeposition of the conductive polymer lawn can further comprise electrodepositing at least a portion of the conductive polymer beneath the porous surface and within pores of the p-doped silicon substrate.

In any of the foregoing embodiments, the p-doped silicon substrate can comprise a dopant selected from the group consisting of boron (preferable), aluminum, gallium, indium, and combinations thereof. The p-doped silicon substrate preferably has a resistivity ranging from about 0.001 ohm-cm or about 0.01 ohm-cm up to about 0.02, 0.05, 0.08, or 0.1 ohm-cm. The p-doped silicon substrate can have a bi-layer structure comprising a crystalline silicon layer adjacent to a porous silicon layer, where the porous silicon layer comprises the porous surface and has a thickness ranging from about 1 nm to about 50 nm, about 2 nm to about 20 nm, or about 5 nm to about 15 nm.

Similarly, the conductive polymer can generally be selected from the group consisting of polyanilines, polyphenylenes, polyphenylene vinylenes, polythiophenes, polypyrroles, polyfurans, polyselenophenes, polyisothianapthenes, polyphenylene sulfides, polyacetylenes, polydiacetylenes, polypyridyl vinylenes, polycarbazoles, conductive carbohydrates, conductive polysaccharides, derivatives thereof, blends thereof with other polymers, copolymers of the monomers thereof, and combinations thereof. Preferably, the conductive polymer comprises one or more of an electrolyte-doped polypyrrole, polyaniline, and polythiophene.

The binding pair member can generally be selected from the group consisting of antibodies, antibody fragments, antigens, biotin, avidin and derivatives thereof, hormones, hormone receptors, polynucleotides, oligonucleotides, aptamers, whole cells, and combinations thereof. Preferably, the binding pair member comprises a probe DNA (pDNA) oligonucleotide. The binding pair member can be electrostatically bound to the conductive polymer in some embodiments.

The composite material according to any of the foregoing embodiments can be used in a biosensor electrode. The biosensor electrode can be provided in a kit for an assay for a target analyte, for instance a kit that additionally includes an electrical detection apparatus and/or reagents to perform the assay. Still further, the composite material can be included in a kit comprising the composite material in a container and reagents to perform an assay for an analyte in a solution which binds the binding pair member. Further still, the disclosure relates to a method and test kit for performing the assay for an analyte which comprises using the composite material.

Previous approaches to forming a silicon substrate-conductive polymer (CP) biosensor require deposition of a metal thin film on the silicon substrate first and a second deposition of the conductive polymer on the metal thin film. The resulting cross-sectional structure is silicon-metal-CP. According to the present disclosure, a metal film is not used. Instead, a porous silicon (PS) surface/layer is formed in a silicon wafer and is used as the contact surface for the conductive polymer. Therefore, the resulting cross-sectional structure is silicon-PS-CP. This enables the deposition of the conductive polymer directly onto a silicon substrate. The conductive polymer can be polymerized by applying an adequate amount of negative current or positive potential, for example by using chronoamperometry, chronopotentiometry, or cyclic voltammetry. The same techniques can be used for porous silicon formation. The amount of current or potential depends on the experimental conditions such as electrolyte concentration, geometry of the electrochemical cell, etching/monomer solution compositions.

All patents, patent applications, government publications, government regulations, and literature references cited in this specification are hereby incorporated herein by reference in their entirety. In case of conflict, the present description, including definitions, will control.

Additional features of the disclosure may become apparent to those skilled in the art from a review of the following detailed description, taken in conjunction with the drawings, examples, and appended claims.

### BRIEF DESCRIPTION OF THE DRAWINGS

For a more complete understanding of the disclosure, reference should be made to the following detailed description and accompanying drawings wherein:

FIGS. 1a-1d illustrate a composite material 10 and a method for making the same according to the disclosure.

FIG. 2 illustrates a biosensor kit 30 including the composite material 10.

FIG. 3 illustrates an electrochemical cell 100 used for the production of porous silicon and electropolymerization of a conductive polymer thereon.

FIG. 4 illustrates an electrochemical system using the electrochemical cell 100 to form porous silicon using a source meter (Keithey Model 2400) as a power source.

FIG. 5 illustrates an electrochemical system using the electrochemical cell 100 to form porous silicon using a potentiostat (Model M263, Princeton Applied Research) interfaced with PC as a power source. RE, WE, and CE denote reference, working and counter electrodes, respectively.

FIG. 6 illustrates an electrochemical system using the electrochemical cell 100 to electropolymerize pyrrole using a potentiostat (Model M263, Princeton Applied Research) interfaced with PC as a power source.

FIG. 7 and inset 7A are graphs illustrating the capacitance-voltage relationship of electropolymerization of 0.02 M pyrrole in acetonitrile containing 0.1 M HCl 04 as a supporting electrolyte. The scan rate was 25 mV-s⁻¹ over 10 cycles of potential scanning.

FIG. 8 is a cross-sectional SEM image of an analyte-bound substrate PS/PPy+pDNA+tDNA). Bulk boron-doped polycrystalline silicon (0.01-0.02 ohm-cm resistivity and [100] orientation) was anodized in an etchant solution (HF:CH₃CH₂OH) with a −5 mA-cm⁻² constant forcing function for 30 minutes to form a porous silicon upper layer. A polypyrrole layer was directly electropolymerized on the porous silicon layer bound to pDNA and tDNA. A 10 nm gold layer was deposited on the top surface for imaging.

FIG. 9 is a plot of iₚ versus tDNA concentration after 1 h of incubation time (closed circle). The iₚ observed at about 0.2 V versus Ag/AgCl were selected for the calibration. Open circles represent background signals obtained from nPS/PPy+tDNA substrates.

FIG. 10 is a plot of iₚ versus incubation time, where the tDNA concentration is 0.909 μM. The iₚ observed at about 0.2 V versus Ag/AgCl were selected for the calibration.

While the disclosed compositions and methods are susceptible of embodiments in various forms, specific embodiments of the disclosure are illustrated in the drawings (and will hereafter be described) with the understanding that the disclosure is intended to be illustrative, and is not intended to limit the claims to the specific embodiments described and illustrated herein.

### DETAILED DESCRIPTION

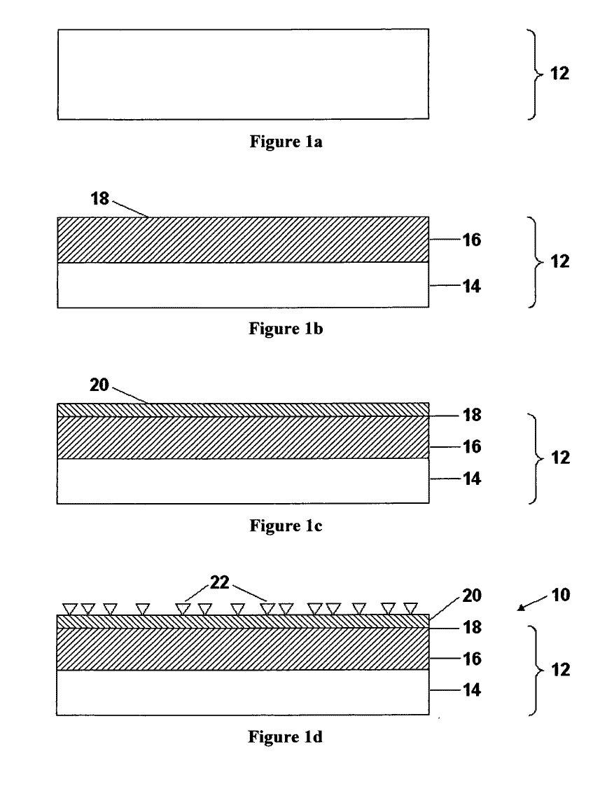

The disclosure relates to porous silicon/conductive polymer composites that can be used in biosensor applications (e.g., in a binding assay that captures a target analyte). With reference to FIGS. 1a-1d, a composite material 10 generally includes (a) a p-doped silicon substrate 12 that has a porous surface 18; (b) a lawn of a conductive polymer 20 bound to the porous surface 18; and (c) a binding pair member 22 bound to the conductive polymer 20. Processes according to the disclosure for forming the composite material 10 generally include electropolymerizing and electropolymerizing the conductive polymer 20 onto the porous surface 18 of the silicon substrate 12 and then binding the binding pair member 22 to the conductive polymer 20.

### Silicon Substrate

The silicon substrate is generally a p-doped silicon substrate that is formed from a p-doped crystalline silicon substrate (e.g., p-doped polycrystalline silicon (p-Si)). The porous surface of the silicon substrate can be formed by etching pores into a surface/external layer of the p-doped crystalline silicon substrate by using an acid etchant solution and by applying an anodizing current to the crystalline silicon substrate in an electrochemical cell. The acid etchant preferably includes hydrofluoric acid, for example a mixture of hydrofluoric acid in ethanol. The porous surface also can be formed using a stain-etching process by exposing a surface of the silicon substrate to a stain etching solution (e.g., an aqueous solution including hydrogen fluoride and nitric acid). The pore formation results in a p-doped silicon substrate 12 having two silicon layers: a crystalline silicon layer 14 adjacent to a porous silicon layer 16 (the outer portion of which is the porous surface 18), for example as illustrated in FIGS. 1a and 1b. Preferably, the porous silicon layer is annealed (e.g., at a temperature of about 70° C. to about 200° C. or about 90° C. to about 150° C.), for example immediately after anodization or prior to electropolymerization, and/or after a silicon oxide layer has formed on the porous surface (e.g., resulting from exposure of the porous surface to air). The depth/thickness of the porous silicon layer depends of the etching conditions (e.g., etching time, acid concentration, anodizing current) and suitably ranges from about 1 μm to about 50 μm, about 2 μm to about 20 μm, or about 5 μm to about 15 μm based on the selected etching conditions.

The porous layer/surface of the silicon substrate provides a rough surface that facilitates the subsequent attachment of an external conductive polymer layer to the silicon substrate without an intervening metallic layer (or a layer of other material). The pores (not shown) are generally sized on the nanometer scale, for example ranging in size (or having an average size) from about 1 nm to about 50 nm, about 2 nm to about 20 nm, or about 5 nm to about 15 nm.

The silicon substrate can be doped with other elements to adjust its electrical response by controlling the number and charge of current carriers in the substrate. The p-doped silicon substrate includes one or more trivalent atoms (typically from Group IIIA of the periodic table) substituted into the crystal silicon lattice, thereby creating electron holes in the normal silicon lattice and increasing the number of free charge carriers in the silicon substrate. Suitable dopant elements include boron (preferable), aluminum, gallium, indium, and combinations thereof. The resulting p-doped silicon substrate is a semiconductor and has a resistivity ranging from about 0.001 ohm-cm or more and/or up to about 1000 ohm-cm, for example from about 0.01 ohm-cm to about 0.1 ohm-cm, about 0.01 ohm-cm to about 0.08 ohm-cm, about 0.01 ohm-cm to about 0.05 ohm-cm, or about 0.01 ohm-cm to about 0.02 ohm-cm.

### Conductive Polymer

The composite material includes a lawn of a conductive polymer bound to the porous surface of the silicon substrate. A polymer “lawn” includes a layer of the conductive polymer that has multiple linear or branched segments of the conductive polymer as a pore-assembled monolayer extending from the surface of the porous silicon. The lawn enables binding pair members to be attached to each segment of the lawn so that the capture of an analyte by a binding pair member is more efficient.

The conductive polymers according to the disclosure are not particularly limited and generally include any polymer that is electrically conductive. Suitable examples of conductive polymers include polypyrrole (preferable), polyaniline, and polythiophene (e.g., poly(3-methylthiophene)), which are dispersible in water and are conductive because of the presence of an electrolyte (e.g., an anion or a cation) doped into the conductive polymer (e.g., resulting from acid-doping of the polymer or monomer). Suitable electrolyte dopants for conductive polymers in general include acid dopants such as perchloric acid and/or hydrochloric acid. Other electrically conductive polymers include substituted (e.g., functional derivatives, for example those with functional groups that permit the grafting of binding pair members to the conductive polymers) and unsubstituted polyanilines, polyphenylenes (e.g., polyparaphenylenes), polyphenylene vinylenes, polythiophenes, polypyrroles, polyfurans, polyselenophenes, polyisothianapthenes, polyphenylene sulfides, polyacetylenes, polydiacetylenes, polypyridyl vinylenes, polycarbazoles, biomaterials, biopolymers, conductive carbohydrates, conductive polysaccharides, combinations thereof and blends thereof with other polymers, copolymers of the monomers thereof. Illustrative are the conductive polymers are described in U.S. Pat. Nos. 6,333,425, 6,333,145, 6,331,356, and 6,315,926. Preferably, the conductive polymers do not contain metals in their metallic form.

The conductive polymer is generally deposited as a lawn 20 (FIG. 1c) on the silicon substrate 12 by electropolymerizing of a conductive polymer monomer (e.g., pyrrole, aniline) in a solution (e.g., aqueous-based or organic-based, for example using acetonitrile as a solvent/polymerization medium) in contact with the porous surface 18 of the silicon substrate 12. The polymerization solution generally includes an electrolyte dopant (e.g., anions or cations) to impart electrical conductivity to the resulting polymer. The polymerization reaction can be initiated by the addition of an oxidant (e.g., ammonium persulfate). Upon completion of the polymerization reaction, the silicon substrate includes a lawn of the conductive polymer bound to the porous surface of the silicon substrate. Preferably, the conductive polymer both (a) penetrates and is deposited within at least a portion of the porous interior of the substrate and (b) forms a layer external to the silicon substrate on the porous surface. The thickness of the conductive polymer lawn 20 generally depends on the deposition conditions (e.g., deposition time, monomer concentration, bias potential), but is suitably about 10 nm or more and/or about 10 μm or less, for example ranging from about 20 nm to about 2 μm, about 50 nm to about 500 nm, or about 100 nm to about 300 nm. The portion of the conductive polymer that is internal to the porous substrate helps anchor the external conductive polymer layer, thereby preventing delamination of the conductive polymer from the silicon substrate.

The conductive polymer can be electropolymerized on the silicon substrate by any suitable method, for example including cyclic voltammetry, chronoamperometry, and/or chronopotentiometry. Cyclic voltammetry includes the use of cycles of voltage and current to electropolymerize the monomers corresponding to the conductive polymer. Typically, there are about 0.01 to 0.2 mole units of the monomer (e.g., pyrrole) per segment. Examples of cyclic voltammetry are disclosed in Alocilja et al. U.S. Pat. No. 6,537,802, Alocilja et al. U.S. Pat. No. 6,767,732, and Alocilja et al U.S. Publication No. 2006/0228738, each of which are incorporated herein by reference in its entirety. Chronoamperometry is an electrochemical technique in which the potential of the working electrode is stepped as a function of time. Chronopotentiometry is an electrochemical technique in which the current is controlled and the rate of change in potential versus time is measured at the working electrode.

The conductive polymer provides a substrate for the subsequent attachment of a binding pair member bound thereto, which binding pair member is complementary to a target analyte and thereby forms the composite material for use in the binding assay (e.g., as part of a biosensor). The electrically conductive characteristics of the conductive polymer also can facilitate detection of an analyte bound to the composite material, for example by measuring the electrical resistance or conductance through the conductive polymer, where the measured electrical resistance or conductance is proportional to the amount of the target analyte bound to the binding pair member (i.e., and thereby immobilized on the composite material).

### Binding Pair Member

The composite material 10 includes a binding pair member 22 bound to the conductive polymer 20 (FIG. 1d). A binding pair refers to a pair of complementary constituents that are capable of binding to each other chemically and/or physically (e.g., proteins). The binding pair member 22 is selected to be complementary to a target analyte (not shown) so that the composite material 10 can be used for the selective detection of the target analyte in an assay sample, for example using a biosensor incorporating the composite material 10.

An analyte (or target analyte) generally includes a chemical or biological material, including living cells, in a sample which is to be detected using the composite material. The analyte can include pathogens of interest, for example bacterial and viral pathogens. Specific examples include various Salmonella species (e.g., Salmonella enteritidis), various E. coli strains (e.g., E. coli O157:H7), B. anthracis, B. cereus, and Helicobacter pylori. The analyte also may be an antigen, an antibody, a ligand (i.e., an organic compound for which a receptor naturally exists or can be prepared, for example one that is mono- or polyepitopic, antigenic, or haptenic), a single compound or plurality of compounds that share at least one common epitopic site, and a receptor (i.e., a compound capable of binding to an epitopic or determinant site of a ligand, for example thyroxine binding globulin, antibodies, enzymes, Fab fragments, lectins, nucleic acids, protein A, complement component Clq). In some embodiments, the term “analyte” also can include an analog of the analyte (i.e., a modified form of the analyte which can compete with the analyte for a receptor) that can also be detected using the composite material.

A sample generally includes an aliquot of any matter containing, or suspected of containing, the target analyte. For example, samples can include biological samples, such as samples from taken from animals (e.g., saliva, whole blood, serum, plasma, urine, tears, and the like), cell cultures, plants; environmental samples (e.g., water); and industrial samples. Samples may be required to be prepared prior to analysis according to the disclosed methods. For example, samples may require extraction, dilution, filtration, centrifugation, and/or stabilization prior to analysis. For the purposes herein, “sample” can refer to either a raw sample as originally collected or a sample resulting from one or more preparation techniques applied to the raw sample.

The binding pair member (or specific binding partner) generally includes one of two different molecules, each having a region or area on its surface or in a cavity that specifically binds to (i.e., is complementary with) a particular spatial and polar organization of the other molecule. The binding pair members can be referenced as a ligand/receptor (or antiligand) pair. These binding pair members include members of an immunological pair such as antigen-antibody. Other specific binding pairs such as biotin-avidin (or derivatives thereof such as streptavidin or neutravidin), hormones-hormone receptors, IgG-protein A, polynucleotide pairs (e.g., DNA-DNA, DNA-RNA), probe-target nucleotide pairs (e.g., oligonucleotide DNA or RNA probe complementary to a target type of DNA or RNA), DNA aptamers, and whole cells are not immunological pairs, but can be used as binding pair members within the context of the present disclosure. A preferred binding pair includes a probe-target nucleotide pair, for example a probe DNA (pDNA) oligonucleotide (e.g., having about 5 to about 200 bases, or about 10 to about 50 bases) that is complementary to a target DNA (tDNA) in the species Salmonella enteritidis (e.g., a pDNA sequence of 5′-[Amino link] AATATGCTGCCTACTGCCCTACGCTT-3′, corresponding to positions 690-716 of target, 26 bases; SEQ ID NO: 3).

Preferably, the binding pair members are specific to each other and are selected such that one binding pair member is the target analyte of interest and the other binding pair member is the constituent bound to the conductive polymer of the particulate composition. Binding specificity (or specific binding) refers to the substantial recognition of a first molecule for a second molecule (i.e., the first and second members of the binding pair), for example a polypeptide and a polyclonal or monoclonal antibody, an antibody fragment (e.g., a Fv, single chain Fv, Fab′, or F(ab′)₂ fragment) specific for the polypeptide, enzyme-substrate interactions, and polynucleotide hybridization interactions. Preferably, the binding pair members exhibit a substantial degree of binding specificity and do not exhibit a substantial amount of non-specific binding (i.e., non-covalent binding between molecules that is relatively independent of the specific structures of the molecules, for example resulting from factors including electrostatic and hydrophobic interactions between molecules).

Substantial binding specificity refers to an amount of specific binding or recognition between molecules in an assay mixture under particular assay conditions. Substantial binding specificity relates to the extent that the first and second members of the binding pair to bind only with each other and do not bind to other interfering molecules that may be present in the analytical sample. The specificity of the first and second binding pair members for each other as compared to potential interfering molecules should be sufficient to allow a meaningful assay to be conducted for the target analyte. The substantial binding specificity can be a function of a particular set of assay conditions, which includes the relative concentrations of the molecules, the time and temperature of an incubation, etc. For example, the reactivity of one binding pair member with an interfering molecule as compared to that with the second binding pair member is preferably less than about 25%, more preferably less than about 10% or about 5%.

A suitable binding pair member is an antibody (an immunoglobulin) that specifically binds to and is thereby defined as complementary with a particular spatial and polar organization of another molecule (e.g., an antigen). Antibodies generally include Y-shaped proteins on the surface of B cells that specifically bind to antigens such as bacteria, viruses, etc. The antibody can be monoclonal or polyclonal and can be prepared by techniques that are well known in the art such as immunization of a host and collection of sera (polyclonal) or by preparing continuous hybrid cell lines and collecting the secreted protein (monoclonal), or by cloning and expressing nucleotide sequences or mutagenized versions thereof coding at least for the amino acid sequences required for specific binding of natural antibodies. Antibodies may include a complete immunoglobulin or fragment thereof, which immunoglobulins include the various classes and isotypes, such as IgA, IgD, IgE, IgG1, IgG2a, IgG2b, IgG3, IgM, etc. Fragments thereof may include Fab, Fv and F(ab′)₂, and Fab′. In addition, aggregates, polymers, and conjugates of immunoglobulins or their fragments can be used where appropriate so long as binding affinity for a particular molecule is maintained.

The binding pair member that is specific to the target analyte can be bound to the conductive polymer of the particulate composition by any of a variety of methods known in the art appropriate for the particular binding pair member (e.g., antibody, DNA oligonucleotide). For example, an ionic or otherwise charged binding pair member (e.g., a probe DNA oligonucleotide) can be electrostatically adsorbed onto the conductive polymer by applying an electrical bias to the conductive polymer (e.g., a positive bias for a probe DNA strand having a negative charge). Similarly, antibodies can be bound to the conductive polymer of the particulate composition by incubating the antibodies in a buffer (e.g., a phosphate buffer at a pH of about 7.4 containing dimethylformamide and lithium chloride) suspension of the particulate composition. Similarly, oligonucleotides can be incubated in a buffer (e.g., an acetate buffer at a pH of about 5.2) suspension of the particulate composition that also includes an immunoconjugating agent (e.g., 1-ethyl-3-(3-dimethylaminopropyl) carbodiimide hydrochloride (“EDAC”)). After a suitable incubation period (i.e., depending on the rate of binding between the binding pair member and the conductive polymer) the resulting BEAM nanoparticles can be blocked, washed, centrifuged, and then stored as a suspension (e.g., in aqueous LiCl for an antibody on a phosphate-buffered saline (“PBS”) solution for an oligonucleotide). The binding pair member also can be covalently bound to the conductive polymer. Specifically, a monomer (e.g., pyrrole) is copolymerized with a functionalized monomer (e.g., functionalized pyrrole), the functional group of which allows grafting of the binding pair member once the functionalized conductive polymer has been deposited on the substrate, for example as described in Alocilja et al. U.S. Publication No. 2006/0228738 (incorporated herein by reference).

An advantage of the disclosed composite material includes the ability to perform label-free assays for the detection of target analytes using the same. A detection label is generally part of a conjugate reagent that includes a label and an analyte-specific binding partner or an analyte analog, for example including enzymes, chromogenic substrates, chromophores, radioisotopes, fluorescent molecules, chemiluminescent molecules, phosphorescent molecules, and/or direct visual labels. However, the binding of the target analyte to the binding pair member of the composite material changes the conductivity of the composite material/conductive polymer by an amount proportional to the amount of the target analyte bound to the binding pair member, thus eliminating the need for a detection label to determine the amount of bound analyte.

### Biosensor

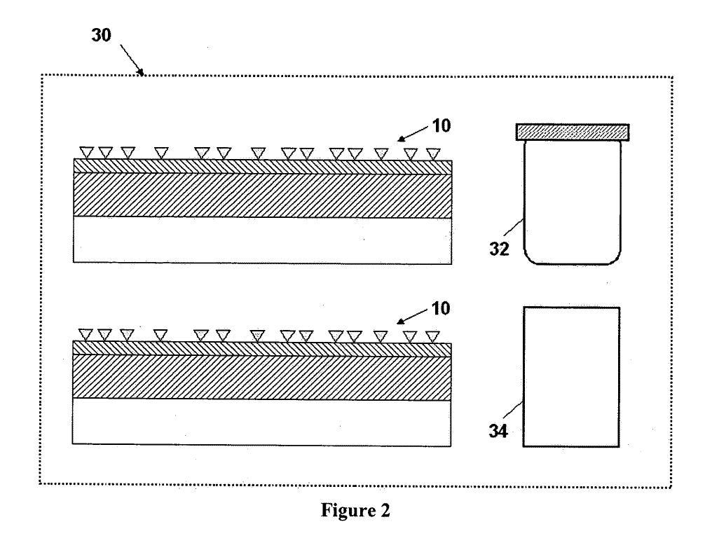

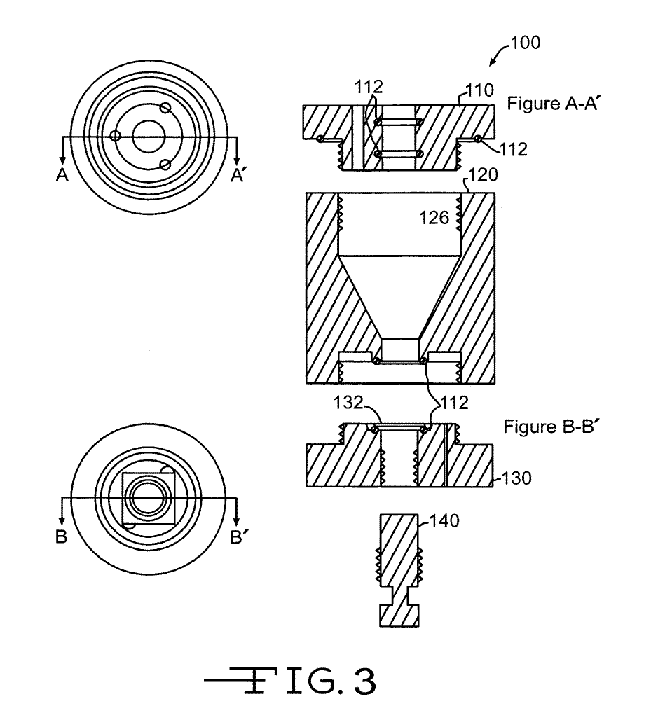

The composite material according to the disclosure can be used in a biosensor electrode. A sample (e.g., a liquid sample) that contains or is suspected of containing a target analyte that is complementary with the composite material's binding pair member can be incubated (e.g., at a predetermined, constant temperature) in contact with the composite material for a predetermined time. After the predetermined time, the conductivity/current across the biosensor can be measured with an electrical detection apparatus (e.g., a potentiostat), and the measured result correlated to the amount of target analyte present in the sample. Thus, the biosensor electrode can be provided in a kit 30 for providing an assay for the target analyte (FIG. 2). For instance, the kit 30 can include one or more composite material units 10 and can additionally include an electrical detection apparatus 34 and/or reagents 32 to perform the assay (e.g., reagents that convert a raw sample into a suitable form for detection by the biosensor, for instance reagents that provide the raw sample in a liquid medium and/or convert the target analyte into a form that suitably binds with the composite material's binding pair member). Preferably, the composite material units 10 in the kit 30 already have the binding pair member 22 bound thereto, and thus are provided in a form that is already specific to the target analyte. In some embodiments, the composite material units 10 may be non-specific (i.e., they do not contain a binding pair member 22), and a user can tailor the kit 30 to the desired target analyte by binding an appropriate binding pair member 22 to the composite material unit 10 prior to analysis. If stored for an extended period (e.g., several days, weeks, months, or more), the kit 30 is preferably stored in a refrigerated environment to preserve the activity/integrity of the composite material units 10 prior to use.

### Example

The following example illustrates the disclosed compositions and methods, but is not intended to limit the scope of any claims thereto.

A porous silicon (PS) structure provides three advantages for biosensor development. A large surface-to-volume ratio and rough surface for biological immobilization can improve the sensitivity when the PS surface is used as a sensing electrode of the sensor module [11]-[13]. Additionally, the optical properties of PS substrate can be explored for the fabrication of optoelectronic devices based on, for example, photoluminescence of PS skeleton and on low-pass IR filtration [14], [15]. To make a sensing electrode active, a binding pair member complementary to a target molecule/analyte should be immobilized on the sensing electrode. The rough surface of PS layer can give extra bonding strength between the binding pair members and the electrode substrate.

A conductive polymer (CP) can play a role as an immobilization matrix for such binding pair members (e.g., enzyme, antibody, DNA), because a positively charged CP matrix can make electrostatic binding to negatively charged biomaterials [16]-[18]. CP films can be commonly synthesized by electropolymerization. Furthermore, the shape, thickness, and conductivity of an electropolymerized CP can be easily modified by adjusting the electrolyte and applied potential. The bonding strength between the CP and the binding pair member is strong enough and comparable to that of covalent bonding in a silanization method. Since electropolymerization is simple and reliable, the application of electropolymerized CP film can be exploited.

In this example, nanoporous silicon (nPS)-based label-free DNA chips were fabricated and functionalized for monitoring Salmonella enterica serovar Enteritidis. A low-resistivity p-Si was electrochemically anodized to obtain the nPS layer. Conductive polypyrrole (PPy) film was directly electropolymerized on top of the nPS as a matrix for probe DNA (pDNA) immobilization and target (tDNA) hybridization steps. PPy has excellent electrical and physicochemical properties in addition to its outstanding compatibility with biomaterials [19]. The peak current output from the label-free DNA chip was used for calibration as a function of hybridization time and tDNA concentration.

Materials: A clinical strain of Salmonella Enteritidis (strain S-64) was grown on trypticase soy agar containing 0.6% yeast extract (TSAYE) and/or broth (TSBYE) at 37° C., as appropriate. In the broth culture, cells were grown to exponential phase, and enumerated by spiral plating appropriately diluted cultures on Bismuth Sulfite Agar and Brilliant Green Agar. The cultures were serially diluted for DNA extraction so that the number of bacterial cells ranged from to 10⁰ to 10⁸ colony forming units per milliliter (CFU/mL). Primers used for polymerase chain reaction (PCR) were designed for the detection of Salmonella Enteritidis from the insertion element (Iel) gene. The single strand forward and reverse primers were IelL-5′-CTAACAGGCGCATACGATCTGACA-3′ (SEQ ID NO: 1; positions 542-565, 24 bases) and IelR-5′-TACGCATAGCGATCTCCTTCGTTG (SEQ ID NO: 2; positions 1047-1024, 24 bases). The Probe DNA (PDNA) used was 5′-[Amino link] AATATGCTGCCTACTGCCCTACGCTT-3′ (SEQ ID NO: 3; positions 690-716 of target, 26 bases). PCR was performed with Taq DNA polymerase in a DNA thermal cycler using a PROMEGA MASTER MIX (Promega Corporation, Madison, Wis.) reaction buffer (pH 8.5), 200 μM deoxynucleotide triphosphates (dNTP), 0.5 μM of the above primers set, 3 mM MgCl₂, and 2.5 U of Taq DNA polymerase. A colony of Salmonella grown overnight in TSAYE was suspended in 0.5 mL of DNA-grade water (Fisher Scientific, Pittsburg, Pa.) containing 5 mM of NaOH and boiled for 10 min to rupture the cells. One μL was added to the PCR reaction mixture. After the final cycle, the target DNA (tDNA) sample was maintained at 72° C. for 10 min to complete the DNA strands synthesis and cooled to 4° C.

A boron-doped, one-side-polished p-Si wafer (0.01-0.02 ohm-cm, 100 orientation, Montco Silicon Technologies, Inc., PA) was employed as a starting material. Pyrrole monomer and 70% solution of HClO₄ (Aldrich Chemical Company, WI) were used for electropolymerization without further purification. Pt wire (99.99%) and an Ag/AgCl double junction probe with porous ceramic wick (Aldrich Chemical Company, WI) were used for counter and reference electrodes, respectively. A MILLI-Q pure water system was used for the preparation of deionized water (D.I. water). Acetonitrile (HPLC grade, EMD Science) was used as a nonaqueous media for the PPy electropolymerization. Highly purified KClO₄ (99.99%) (Aldrich Chemical Company, WI) was used as a supporting electrolyte of all aqueous media. Ethanol (>99.5%, Aldrich Chemical Company, WI) was used for etchant preparation and provisional storage of PS substrate before annealing. A 10 M NaOH solution (Spectrum Quality Products, Inc., CA) was used for stabilizing pDNA. All types of glasswares were washed in either acidic or basic cleaning solution for at least 24 h before use. All reagents were used without further purification.

Electrochemical Cell: FIGS. 3 to 6 illustrate an electrochemical cell 100 that was used in the following examples for both anodization of a silicon substrate and electropolymerization of a conductive polymer (polypyrrole in this case). The electrochemical cell 100 includes four primary components: a cover 110, a body 120, a bottom 130 and a screw 140 (e.g., made from copper) for electrical contact. The electrochemical cell 100 used had an outer diameter of about 60 mm, an assembled length of about 80 mm, and was generally constructed from TEFLON, with the exception of the conducting screw 140. O-rings 112 (e.g., VITON O-rings) were incorporated into the electrochemical cell 110 at various locations to ensure proper sealing of the electrochemical cell 110 upon assembly.

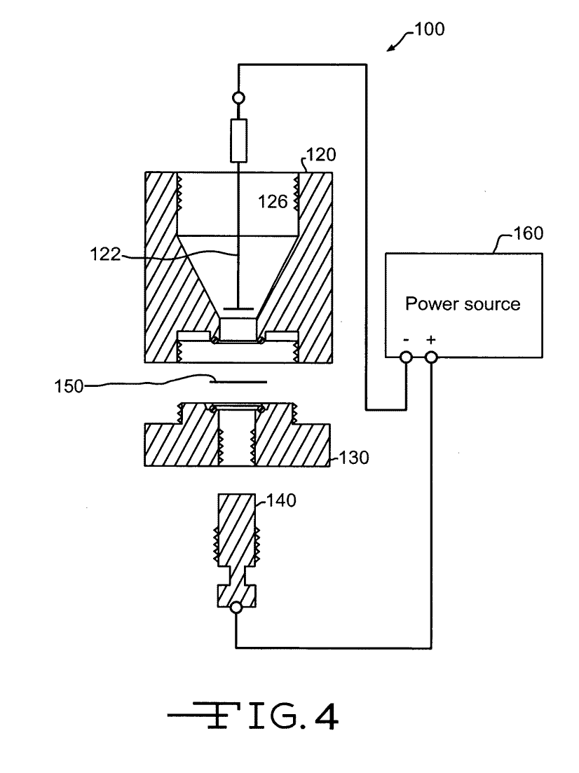

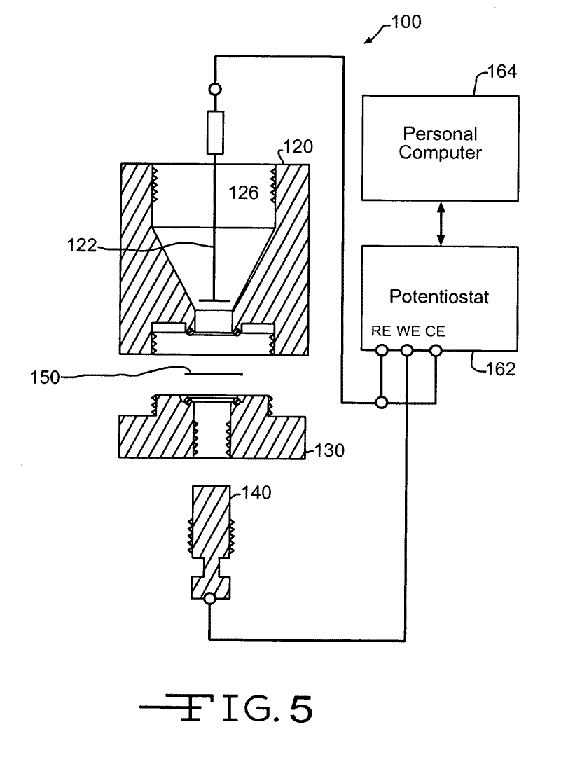

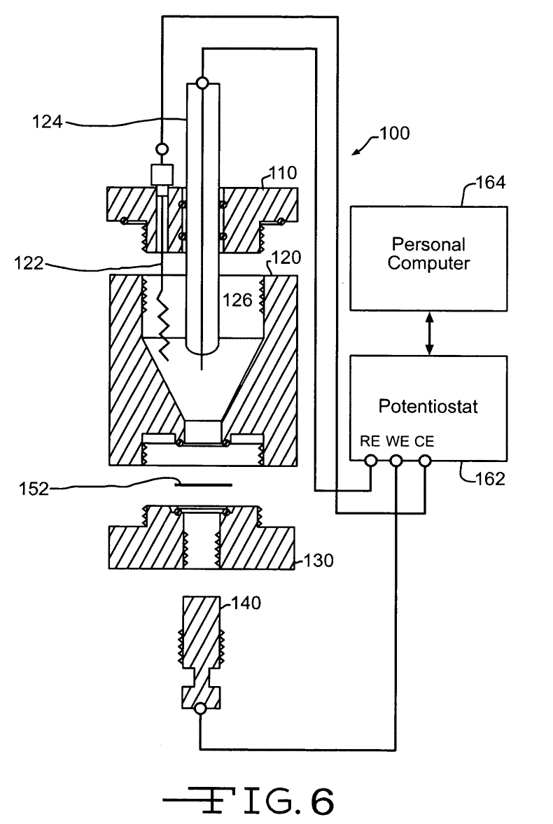

When anodizing a silicon wafer 150 to form a porous silicon substrate 152, only the body 120, bottom 130, and screw 140 components are assembled, for example as illustrated in FIGS. 2 and 3. The silicon wafer 150 is placed into a seat/groove 132, and then the body 120 and bottom 130 are assembled. The screw 140 is inserted into the bottom 130 to provide electrical contact with the silicon wafer 150. An etching solution (e.g., hydrofluoric acid with ethanol and/or water) is as added to a reservoir 126 in the body 120. A platinum counter electrode 122 is dipped into the etching solution and the position of the counter electrode 122 is adjusted to be relatively close to the silicon wafer 150 (e.g., a distance of about 5 mm between the two). In one embodiment (FIG. 4), a power source 160 has a negative lead in electrical connection with the counter electrode 122 and a positive lead in electrical connection with the screw 140. In another embodiment (FIG. 5), a potentiostat 162 interfaced with a computer 164 (e.g., via a GPIB interface) acts as a power source. The potentiostat 162 includes a reference electrode RE and a control electrode CE, both in electrical connection with the counter electrode 122. The potentiostat 162 also includes a working electrode WE in electrical connection with the screw 140.

When electropolymerizing/electrodepositing a conductive polymer on the porous silicon substrate 152, all four components of the electrochemical cell 100 are assembled, for example as illustrated in FIG. 6. The porous silicon substrate 152 remains in the seat/groove 132. A monomer solution (e.g., pyrrole solution containing an electrolyte dopant) is added to the reservoir 126 in the body 120. In this case, the platinum counter electrode 122 and a platinum quasireference electrode 124 are seated in the cover 110 and extend into the monomer solution. The electrodes of the potentiostat 162 are electrically connected as follows: the reference electrode RE with the counter electrode 122, the control electrode CE with the platinum quasireference electrode 124, and the working electrode WE with the screw 140.

Silicon Substrate: A boron-doped, polycrystalline silicon (p-Si) wafer of [100] orientation was cut to an appropriate size and was rinsed and cleaned with 48% HF solution and ethanol to remove any natural oxide layer thereon. The concentration of HF was not a crucial factor. However, a highly concentrated HF solution expedites the oxide removal step. The size of the wafer was selected depending on the size and shape of the electrochemical cell used for anodization and electropolymerization. Silicon wafers of about 19 mm×19 mm×0.5 mm were used in the following examples.

Anodization: A porous silicon (PS) layer was formed on p-Si wafers by electrochemical etching in an etchant solution, a mixture of hydrofluoric acid and ethanol. Etchant composition, elapsed time, and forcing function were varied to evaluate their effect on porous silicon formation. The resistivity of silicon substrates, together with their orientations and doping types, was also an important variable for optimizing porous silicon formation. Before applying an anodizing current, all silicon substrates were cleaned in 48% HF solution for several seconds and their resistivity was measured. Low-resistivity substrates (i.e., ranging from about 0.01 ohm-com to about 0.02 ohm-cm) were used, with specific values ranging from 0.0108-0.0111 ohm-cm for 0.511 mm-thick silicon substrates. Porous silicon formation accompanies the evolution of two hydrogen atoms for each silicon atom dissolved, which means that the current efficiency is about two electrons per dissolved silicon atom. If the current efficiency exceeds four electrons, an electropolishing process (i.e., instead of porous silicon formation), would occur and no hydrogen gas evolution would be observed [1]. Therefore, an objective of porous silicon formation was to minimize electropolishing. The results showed that boron-doped p-Si wafers (having a resistivity of about 0.01 ohm-cm) using an ethanolic HF solution (i.e., HF:CH₃CH₂OH composition with a 3:7 volume ratio of 48% HF and 100% ethanol) as an etchant composition, 30 minutes of time applied, and −5 mA-cm⁻² of forcing function had the best pore formation at room temperature. The area of the working electrode was 1.5 cm² (about 1.2 cm diameter) and compliant voltage required was between 0.42 V and 0.47 V. Additionally, porous silicon substrates formed by current control, instead of being controlled by anodizing bias, showed a more homogeneous surface. Formation of the nPS layer was confirmed by observing visible light emission from the nPS surface when the PS surface was excited with a UV light (254 nm). Because hydrogen evolution was also observed during porous silicon formation, the porous silicon formation mechanism was within the desired current efficiency. Generally, aqueous environments caused structural alteration of as-prepared porous silicon layer to easily change effective optical thickness (EOT) [2].

Porous silicon (PS) is very sensitive to atmospheric conditions and is readily oxidized to silicon dioxide. This oxide layer thickness would grow over time, and hence would affect the emission property of the PS surface with time and, consequently, the electrochemical property of the PPy film on the PS surface would be altered. To minimize uncontrolled oxide formation and to reduce EOT shift caused by environmental exposure, the nPS substrate was annealed. The annealed nPS substrates were stored in a vacuum desiccator before use. One hour of annealing at 110° C. (multiblock heater; Lab-Line Instruments, Inc., IL, USA) was employed to prevent as-prepared porous silicon samples from being oxidized (i.e., a silicon oxide layer was allowed to form in advance and then essentially stopped). All porous silicon substrates were stored in pure ethanol solvent before annealing to prevent the surface from being oxidized in atmospheric conditions. Because polar liquids or vapors can increase the conductivity of a porous silicon layer by orders of magnitude, porous silicon surface was well dried with inert gas before performing resistivity measurements [3], [4]. The resistivity of the annealed porous silicon surface was about 50 ks-cm. This value was several orders larger in magnitude compared to bare silicon substrates with meso- or micro-porous silicon structures [5].

Low-resistivity silicon wafers (about 0.01 ohm-cm to about 0.02 ohm-cm) were selected as a starting material to achieve a better electrical current flow. For comparison, two other types of PS layers were also formed under the same conditions as above (i.e., 30 minutes of etching with ethanolic HF at −5 mA-cm⁻²), but with different resistivities ranging from 14 ohm-cm to 17 ohm-cm and from 0.1 ohm-cm to 0.9 ohm-cm. While the former showed a macro PS layer, the latter was overpolished. The major role of the PS layer is to increase the surface roughness for direct PPy electropolymerization. The nPS formed by using the Low-resistivity p-Si chip with gave better results for the electropolymerized PPy film on the PS substrate than the other two types.

Electropolymerization: A conductive polymer film (polypyrrole (PPy) in this case) for the immobilization of a pDNA binding pair member was formed on porous silicon substrates by anodical electropolymerization (scanning a linear potential with a scan rate of 25 mV-s⁻¹; Princeton Applied Research, Model 263A) in an organic medium: acetonitrile containing 0.02 M pyrrole monomer and 0.1 M perchloric acid. A platinum tip and wire were used for quasireference electrode (QRE) and counter electrode, respectively. Nitrogen gas was bubbled into the 5 mL electrolyte monomer solution for 10 minutes and purged during each of the 10 cycles of potential scanning. Generally, polypyrrole electropolymerization can be achieved at less than 1 V of bias potential with noble metal electrodes. However, the silicon substrates used here are bare, absent of any metallic thin film deposition. Even though the resistivity of the boron-doped p-Si substrates used in the examples was much lower than that of commonly employed Si substrates, it was still too high compared with those of noble metals, which generally range from about 10⁻⁵ to 10⁻⁶ ohm-cm. Because a porous silicon structure has intrinsically high resistivity, the porous silicon layer itself can act as a passivation layer [6]. Therefore, a much higher driving force was required for the initiation of electropolymerization on a porous silicon substrate.

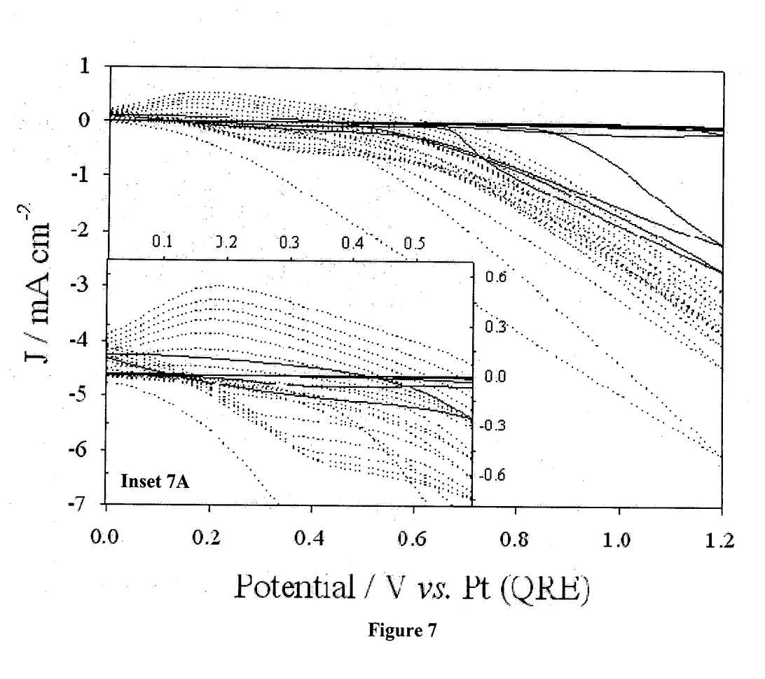

FIGS. 7 and 7A (inset) show capacitance-voltage (CV) diagrams of polypyrrole electropolymerization on planar silicon (PLS, solid lines) and porous silicon PS (dotted lines) substrates. Perchloric acid was used as a supporting electrolyte. When a positive potential was applied, perchlorate anions moved in from the electrolyte phase to the delocalized charge sites of polypyrrole (i.e., p-type doping of the polypyrrole occurs). On polypyrrole electropolymerization, perchlorate anions can give the maximum doping level of 33%, which means 33 dopants are doped for every one hundred monomer units [7]. Generally, increased doping levels lead to increased conductivity, even though doping may not be uniform. As shown in FIGS. 7 and 7A, current increases with increasing number of potential scans when the bias potential is brought up to 1.2 V vs. Pt (QRE) (i.e., the voltage difference between the counter electrode and the quasireference electrode). The inset shows CV diagrams from 0 V to 0.5 V of scan potential.

Enhanced capacitive current (i_{ca}) flow was observed with potential scan between 0.1-0.5 V vs. Pt (QRE). However, both plain silicon (PLS) and porous silicon (PS) substrates had different CV characteristics. The former displayed that the i_{ca} stopped increasing from the third scan and decreased dramatically thereafter, while the i_{ca} of the porous silicon substrate kept increasing. An increase in i_{ca} meant a polymeric film was being formed. A decrease in i_{ca} implied that the polymerization process had stopped and an electroinactive film had formed. Another point of concern in a porous silicon substrate was that current flow in a polymerizing potential region attenuated gradually with an increasing potential scanning even though the i_{ca} still increased at the same time. The i_{ca} of the plain silicon substrate stopped increasing after merely two (2) potential scans. Because a plain silicon substrate had a polished planar surface, it achieved an electroinactive state earlier than the porous silicon. Once the surface was coated with a dense electroinactive film, a steep decrease in the i_{ca} occurred simultaneously with a reduction of current flow in the polymerizing potential region. However, when the porous silicon substrate had a rough surface and a large surface area to be coated, an electroinactive film was obtained within a short time. The large and rough electrochemical effective areas of the porous silicon substrate made the polypyrrole film deposited on the porous silicon substrate less likely to be turned electroinactive. Therefore, if the upper limit of bias potential or elapsed time, i.e., the number of potential scans were increased, a decrease in the i_{ca} of a porous silicon substrate was eventually observed. For example, when the number of potential scans was increased from 10 to 25, the i_{ca} almost stopped increasing after the ninth scan simultaneously with the decrease of current flow at polymerizing potential slightly and continuously with each potential scan. If the driving force of electropolymerization was enhanced, the electroinactivation process was observed earlier. Actually, the upper limit of bias potential was brought up to 1.7 V vs. Pt (QRE), which means unilateral expansion of polymerizing potential, electroinactive film formation occurred after the sixth scan. This implied that increased bias potential can cause the conductive polypyrrole film to become electroinactive faster. Depletion of pyrrole monomers in the diffusion zone around porous silicon layers by applying high bias potential led to unbalanced potential distribution at the electrical double layer and deteriorated the film conductivity due to overoxidation process. In the meantime, the scan rate was another parameter that affected polypyrrole formation on porous silicon substrates. Basically, oxidation of pyrrole monomers to produce radical-cations is the first step and this was also the rate-determining step in polypyrrole electropolymerization mechanism, i.e., a sufficient amount of monomer radicals formed in a polymerizing potential range to enhance the formation of radical-cation in the next step. When the actual scan rate was doubled from 25 to 50 mV-s⁻¹, appreciable amounts of the i_{ca} increases were perceived from the tenth scan and it stabilized after the seventeenth scan. This implied that about 2 times potential scanning were required at scan rate of 50 mV-s⁻¹; the i_{ca} increase stopped around the ninth scan at 25 mV-s⁻¹. Because the resistivity of both polypyrrole films formed at 25 and 50 mV-s⁻¹ showed the same values as 200 kΩ-cm under well dried conditions, it was deduced that direct electropolymerization of pyrrole on a porous silicon substrate was only a function of applied energy and independent of scan rate. Generally, most conductive polymers (CPs) have a resistivity ranging from about 0.02-200 ohm-cm, and 107 ohm-cm was used as a reference point for setting a conductive polymer as a semiconductor or an insulator [8]. Therefore, it is hard to say that directly electropolymerized polypyrrole films on porous silicon substrates were either electroinactive [9] or electroactive. Moreno et al. reported that polypyrrole-impregnated porous silicon showed 104 times larger conductivity than that of bare porous silicon [10]. This is true for wet samples. However, for a well-dried sample, it was rather reduced; the resistivity of annealed porous silicon was about 50 kΩ-cm compared with about 200 kΩ-cm for porous silicon/polypyrrole. Given experimental conditions, the surface of polypyrrole-coated porous silicon substrates had a dark yellow or green color. This implied that the porous silicon-based polypyrrole film was neither too thick nor too thin, qualitatively.

As a result of using a porous silicon substrate, the adsorption strength between the silicon substrate and the polypyrrole conductive polymer was substantially increased. Even though a hydrophilic polypyrrole film can be formed on hydrophobic silicon surface in nonaqueous media, it will readily detach from a non-porous silicon substrate in aqueous media. A natural oxide layer (i.e., hydrophilic SiO₂) grows more easily on a porous silicon substrate than on the normal silicon substrate (e.g., non-porous or otherwise smooth), and the presence of the natural oxide layer can increase the surface energy between the substrate and conductive polymer, thereby enhancing the adsorption strength between the two composite constituents.

Binding Pair Member: The porous silicon-based conductive polymeric lawn provides an immobilizing/binding matrix for binding pair members (e.g., enzymes, DNA/RNA polynucleotides and oligonucleotides, antibodies), thus forming a composite material that can be suitable used as a part of a biosensor electrode. For example, the intrinsic negative charge of the backbone of a DNA strand was exploited to bind probe DNA (pDNA) to the polypyrrole film by applying a positive bias to the polymer-coated porous silicon substrate in the electrochemical cell, thereby electrostatically adsorbing the pDNA to the conductive polymer lawn and providing a suitable biosensor composite material for monitoring Salmonella enteritidis, which contains target DNA (tDNA) that is complementary to and will bind with the hybridzed pDNA in the composite material.

The positively charged black PPy film is an excellent doping matrix for the negatively charged pDNA. Thus, a solution of 10 nM pDNA was immobilized on the PPy matrix by applying 0.6 V versus Pt (QRE) to the PPy-coated PS substrate (p-Si/PS/PPy) for 20 min. The pDNA had the following 26 base pair sequence: 5′-[Amino link] AATATGCTGCCTACTGCCCTACGCTT-3′ (SEQ. ID NO. 3; corresponding to positions 690-716 of the target). Even though a 10 nM pDNA solution for doping pDNA into the polypyrrole matrix was used, the actual concentration of the pDNA in the polypyrrole film could not be determined. However, it was assumed that the amount of pDNA immobilized on various sensors was identical because the same constant potential was applied over the same time periods.

A hybridization reaction with the tDNA also occurs on the pDNA-doped p-Si/nPS/PPy substrate (p-Si/nPS/PPy/pDNA). Double-stranded tDNA from the target organism prepared earlier was mixed with a hybridization buffer (QUIKHYB, Stratagene) and preheated to 95° C. for 20 min in a water bath, and again cooled to 59° C. (i.e., the melting temperature of pDNA). The hybridization of complementary DNA strands extracted from Salmonella Enteritidis with the pDNA layer was performed in a water bath (ISOTEMP 288, Fisher Scientific) by dropping a mixture of tDNA and hybridization buffer on the pDNA-PS substrate. After hybridization, the surface was thoroughly rinsed with a washing solution composed of 0.1× saline-sodium citrate (SSC) buffer and 0.1% (w/v) of sodium dodecyl sulfate (SDS) to remove nonspecifically adsorbed DNA strands. After rinsing again with a 0.1×SSC buffer, the tDNA-hybridized PS chip was dried under nitrogen and prepared for measurement. Peak current outputs obtained from cyclic voltammograms of each PS chip in a solution containing 0.01 M potassium perchlorate were recorded for calibration of the chip.

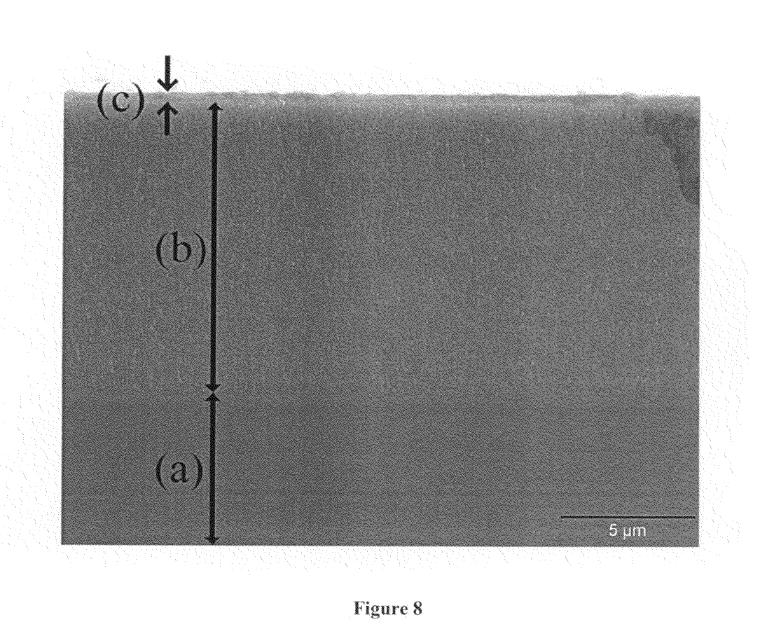

A scanning electron microscopy (SEM; JSM-6300F Scanning Microscope, JEOL) image of the cross-section of the analyte-bound substrate illustrates the successful electropolymerization and attachment of polypyrrole to a porous silicon substrate, and further illustrates the functionalization of the polypyrrole to form a DNA biosensor used to test for Salmonella enteritidis. FIG. 8 illustrates the cross-section of the p-Si/nPS/PPy+pDNA+tDNA multilayered structure (additionally including a 10 nm gold imaging layer predeposited with an SC500, Emscope sputter). The cross-section labeled “a” is the low-resistivity polycrystalline silicon region that remains from the original silicon wafer (i.e., the region was not etched or otherwise converted to a porous structure). The cross-section labeled “b” is the porous silicon layer resulting from etching in which columnar micro pores are distributed homogeneously. The porous layer depth was about 12 μm and the pore diameter was about 10 nm. A very high roughness of the porous silicon substrate allowed the direct electropolymerization of the polypyrrole on the porous silicon substrate. The cross-section labeled “c” is the polypyrrole film that was directly electropolymerized on the rough porous silicon substrate with thickness of about 200 nm. The surface energy of the resulting silicon substrate was enhanced by forming the porous silicon structure and allowed both high quality and sensitive measurements of porous silicon-based DNA sensors in an aqueous media.

Biosensor Calibration: There are various reports about DNA conductivity, from a superconductor to an insulator [20]. Some studies have indicated that DNA is electrically insulator when the length of DNA strand is longer than a few nanometers [21]. Considering that the length of a 10-base-pair DNA is about 3.4 nm, a 26-base-pair DNA is about 8.84 nm long. A PPy film is a semiconductor, and the level of a dopant will strongly affect the resistivity of PPy film (i.e., insulating DNA strands doped to PPy can potentially increase the depleted region of PPy and, thus, conductivity would decrease).

The dependence of peak current (iₚ) around 0.2 V vs. Ag/AgCl on tDNA concentration and incubation time were determined from the cyclic voltammograms of analyte-bound substrates in a 0.01 M potassium perchlorate solution. The analyte-bound substrates include the porous silicon-polypyrrole-pDNA composite material that had bound tDNA as a result of incubation of the composite material with the tDNA sample (i.e., a PS/PPy+pDNA+tDNA multilayered structure was formed).

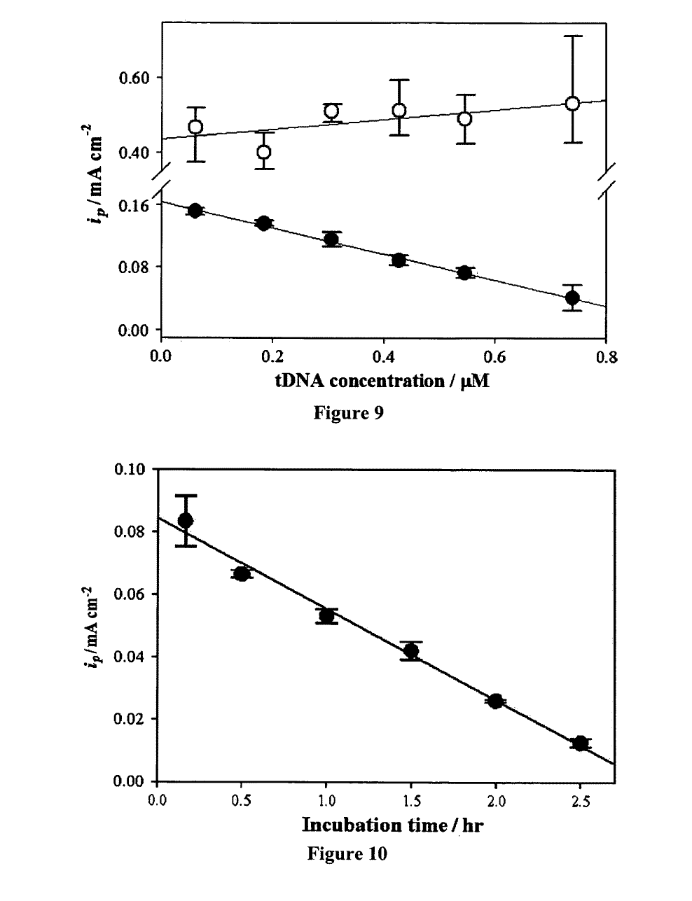

FIG. 9 shows the change with various tDNA concentrations after 1 h of incubation time for tDNA hybridization. The iₚ observed at about 0.2 V versus Ag/AgCl in a CV diagram was selected for the calibration. The iₚ decreased with increasing tDNA concentration due to the decreasing conductivity of the PPy matrix (closed symbols in FIG. 9). The sensitivity determined from the plot of iₚ vs. tDNA concentration was −166.6 μA-cm⁻²-μM⁻¹ within the experimental range of tDNA concentrations tested. A bare PPy film without a pDNA dopant was also used for recording background signals (open symbols in FIG. 9). Since the conductive PPy film is a semiconductor, its conductivity depends on the doping level. Single- and double-stranded DNA can act as a dopant. However, they are electrical insulators compared with normal anions or cations composing an electrolyte. When a tDNA hybridizes to its complementary pDNA, some anions that have been doped into the PPy matrix will be repelled out of the PPy film for neutralization. If there is no hybridization process at all, the conductivity of the PPy film will not change. However, some ions dissolved in a hybridization buffer solution can still move in the PPy film at 59° C. electrolyte phase. The lowest detection limit was 0.167 μM for the experimental tDNA range tested.

FIG. 10 shows the dependence of iₚ on the incubation time for tDNA hybridization. The current density decreased with an increase in incubation time. The iₚ at 0.2 V versus Ag/AgCl was plotted as a function of incubation time and the slope showed a current density (J) decrease by about 29 μA-cm⁻² every hour.

Summary: Thus, label-free DNA sensors based on a porous silicon (PS) substrate were fabricated and electrochemically characterized. A nanoporous silicon (nPS) layer was electrochemically formed on a p-doped p-Si wafer for the direct electropolymerization of pyrrole on the p-Si substrate without predeposition of a metallic thin film. For better electrical conductivity, a low-resistivity p-Si wafer (about 0.01 to 0.02 ohm-cm) was used as starting material. The nPS layer enhanced the surface roughness of the bare p-Si substrate and modified the physicochemical propertied of the p-Si surface. The average size of the nPS layer grown on the p-Si substrate by anodizing and applying −5 mA-cm⁻² of current J in an etchant (HF:CH₃CH₂OH in a 3:7 volume ratio) was 12 μm in depth with pores of about 10 nm in diameter. The formation of a natural oxide layer is easier on an nPS layer than a macro PS. Thus, a conductive PPy film could be directly deposited/electropolymerized on the nPS layer due to a higher amount of SiO₂ formation (i.e., as compared to bare silicon). The rough surface of PS layer enhanced the surface energy of the PS layer to form a stronger adsorption bond and adhesion between nPS and PPy. Electrostatic adsorption of pDNA to the conductive PPy matrix did not require any complicated fabrication procedure unlike the silanization process ([22], [23]), but nonetheless provided a comparable bonding strength. The sensitivity obtained from the plot of iₚ versus tDNA concentration was −166.6 μA-cm⁻²-μM⁻¹.

The nPS-based DNA sensor used the iₚ output to determine the amount of tDNA present in a sample. The optical properties of the PS layer depend on the size of the PS layer [1]. This means that the PS structure strongly affects optoelectronic signal output, but has relatively less effect on the electrochemical signal. Therefore, better reproducibility in the nPS-based electrochemical sensor could be expected. The whole procedure of the nPS-based electrochemical sensor for monitoring pathogens is simple, and rapid detection is feasible compared with the other methods (e.g., impedimetric or spectroscopic methods) [23]. Additionally, label-free sensors are more comprehensive and easier to fabricate than a labeled-DNA sensor in which the DNA strand is conjugated with an electrochemically active reagent that requires complex mechanism to detect. Many works have been done for label-free detection or fabrication of PPy-based biosensor, but this is the first report on the use of PPy-based label-free biosensor for monitoring Salmonella spp.

Because other modifications and changes varied to fit particular operating requirements and environments will be apparent to those skilled in the art, the disclosure is not considered limited to the example chosen for purposes of illustration, and covers all changes and modifications which do not constitute departures from the true spirit and scope of this disclosure.

Accordingly, the foregoing description is given for clearness of understanding only, and no unnecessary limitations should be understood therefrom, as modifications within the scope of the disclosure may be apparent to those having ordinary skill in the art.

Throughout the specification, where the compositions, processes, or apparatus are described as including components, steps, or materials, it is contemplated that the compositions, processes, or apparatus can also comprise, consist essentially of, or consist of, any combination of the recited components or materials, unless described otherwise. Combinations of components are contemplated to include homogeneous and/or heterogeneous mixtures, as would be understood by a person of ordinary skill in the art in view of the foregoing disclosure.

### REFERENCES

1. O. Bisi, S. Ossicini and L. Pavesi, “Porous silicon: a quantum sponge structure for silicon based optoelectronics”, Surface Science Reports, 38, 1-126 (2000).
2. C. Steinem, A. Janshoff, V. S.-Y. Lin, N. H. Volcker and M. R. Ghadiri, “DNA hybridization-enhanced porous silicon corrosion: mechanistic investigations and prospect for optical interferometric biosensing”, Tetrahedron, 60, 11259-11267 (2004).
3. R. Tsu and D. Babic, “Doping of a quantum dot”, Applied Physics Letters, 64 1806 (1994).
4. M. Ben-Chorin, A. Kux and I. Schechter, “Adsorbate effects on photoluminescence and electrical conductivity of porous silicon”, Applied Physics Letters, 64 481 (1994).
5. V. Lehmann, F. Hofmann, F. Moller and U. Gruning, “Resistivity of porous silicon: a surface effect”, Thin Solid Films, 255, 20-22 (1995).
6. A. O. Konstantinov, C. I. Harris, A. Henry and E., Janzen, “Fabrication and properties of high-resistivity porous silicon carbide for SiC power device passivation”, Materials Science and Engineering, B29 114 (1995).
7. P. Chandrasekhar, “Conducting Polymers, Fundamentals and Applications: A Practical Approach”, Kluwer Academic Publishers, (1999).
8. T. A. Skotheim, R. L. Elsenbaumer and J. R. Reynolds, “Handbook of Conducting Polymers”, Second edition, Revised and Expanded.
9. T. Osaka, T. Fukuda, H. Kanagawa, T. Momma and S. Yamauchi, “Application of electroinactive polypyrrole film to the pH sensor electrode”, Sensors and Actuators B, 13, 205-208 (1993).
10. J. D. Moreno, M. L. Marcos, F. Agullo-Rueda, R. Guerrero-Lemus, R. J. Martin-Palma, J. M. Martinez-Duart and J. Gonzalez-Velasco, “A galvanostatic study of the electrodeposition of polypyrrole into porous silicon”, Thin Solid Films 348, 152-156 (1999).
11. E. Hanein, Y. V. Pan, B. D. Ratner, D. D. Denton, and K. F. Böhringer, “Micromachining of non-fouling coatings for bio-MEMS applications,” Sens. Actuators B: Chem., 81: 49-54 (2001).
12. F. P. Mathew and E. C. Alocilja, “Porous silicon-based biosensor for pathogen detection,” Biosens. Bioelectron., 20: 1656-1661 (2005).
13. J.-H. Jin, N. K. Min, and S.-I. Hong, “Poly(3-methylthiophene)-based porous silicon substrates as a urea-sensitive electrode,” Appl. Surf. Sci., 252: 7397-7406 (2006).
14. N. K. Min, C. G. Kang, J.-H. Jin, J. Y. Ko, and S. K. Kim, “Porous silicon-based UV detector,” J. Korean Phys. Soc., 39: S63-S66 (2001).
15. V. Lehmann, R. Detemple, and W. Theiss, “Optical shortpass filters based on macroporous silicon,” Appl. Phys. Lett., 78: 589-591 (2001).
16. E. Llaudet, N. P. Botting, J. A. Crayston, and N. Dale, “A three-enzyme microelectrode sensor for detecting purine release from central nervous system,” Biosens. Bioelectron., 18: 43-52 (2003).
17. C. Tlili, H. Korri-Youssoufi, L. Ponsonnet, C. Martelet, and N. J. Jaffrezic-Renault, “Electrochemical impedance probing of DNA hybridization on oligonucleotide-functionalised polypyrrole,” Talanta, 68: 131-137 (2005).
18. X. Jiang and X. Lin, “Overoxidized polypyrrole film directed DNA immobilization for construction of electrochemical micro-biosensors and simultaneous determination of serotonin and dopamine,” Anal. Chim. Acta, 537: 145-151 (2005).
19. A. Ramanavicius, A. Ramanaviciene, and A. Malinauskas, “Electrochemical sensors based on conducting polymer-polypyrrole,” Electrochim. Acta, 51: 6025-6037 (2006).
20. R. O. Endres, D. L. Cox, and R. R. P. Singh, “Colloquium: The quest for high-conductance DNA,” Rev. Mod. Phys., 76: 195-214 (2004).
21. Y. Ye, L. Chen, X. Liu, and U. J. Krull, “DNA and microfluidics: Building molecular electronics systems,” Anal. Chim. Acta, 568: 138-145 (2006).
22. S. Chan, Y. Li, L. J. Rothberg, B. L. Miller, and P. M. Fauchet, “Nanoscale silicon microcavities for biosensing,” Mat. Sci. Eng. C-bio S., 15: 277-282 (2001).
23. F. Bessueille, V. Dugas, V. Vikulov, J. P. Cloarec, E. Souteyrand, and J. R. Martin, “Assessment of porous silicon substrate for well-characterised sensitive DNA chip implement,” Biosens. Bioelectron., 21: 908-916 (2005).

What is claimed is:

1. A composite material for use in a binding assay that captures an analyte, the composite material comprising:
(a) a p-doped silicon substrate comprising a porous surface;
(b) a lawn of a conductive polymer bound to the porous surface; and
(c) a binding pair member bound to the conductive polymer.

2. The composite material of claim 1, wherein the p-doped silicon substrate comprises a dopant selected from the group consisting of boron, aluminum, gallium, indium, and combinations thereof.

3. The composite material of claim 1, wherein the p-doped silicon substrate has a resistivity ranging from about 0.01 ohm-cm to about 0.08 ohm-cm.

4. The composite material of claim 1, wherein the p-doped silicon substrate comprises a crystalline silicon layer adjacent to a porous silicon layer, the porous silicon layer comprising the porous surface and having a thickness ranging from about 2 μm to about 20 μm.

5. The composite material of claim 1, wherein the conductive polymer is selected from the group consisting of polyanilines, polyphenylenes, polyphenylene vinylenes, polythiophenes, polypyrroles, polyfurans, polyselenophenes, polyisothianapthenes, polyphenylene sulfides, polyacetylenes, polydiacetylenes, polypyridyl vinylenes, polycarbazoles, conductive carbohydrates, conductive polysaccharides, derivatives thereof, blends thereof with other polymers, copolymers of the monomers thereof, and combinations thereof.

6. The composite material of claim 1, wherein the conductive polymer comprises one or more of an electrolyte-doped polypyrrole, polyaniline, and polythiophene.

7. The composite material of claim 1, wherein the binding pair member is selected from the group consisting of antibodies, antibody fragments, antigens, biotin, avidin and derivatives thereof, hormones, hormone receptors, polynucleotides, oligonucleotides, aptamers, whole cells, and combinations thereof.

8. The composite material of claim 1, wherein the binding pair member comprises a probe DNA (pDNA) oligonucleotide.

9. The composite material of claim 1, wherein the binding pair member is electrostatically bound to the conductive polymer.

10. A kit comprising:
(a) a biosensor electrode comprising the composite material of claim 1; and
(b) reagents to perform an assay for an analyte that is complementary to the binding pair member of the composite material.

11. A composite material for use in a binding assay that captures an analyte, the composite material comprising:
(a) a p-doped silicon substrate comprising a porous surface and a boron dopant;
(b) a lawn of a conductive polymer bound to the porous surface, the conductive polymer comprising one or more of an electrolyte-doped polypyrrole, polyaniline, and polythiophene; and
(c) a probe DNA (pDNA) oligonucleotide electrostatically bound to the conductive polymer.

12. A process for forming a composite material for use in a binding assay that captures an analyte, the process comprising:
(a) providing a p-doped silicon substrate comprising a porous surface;
(b) electrodepositing a lawn of a conductive polymer onto to the porous surface; and
(c) binding a binding pair member to the conductive polymer, thereby forming the composite material.

13. The process of claim 12, wherein part (a) further comprises:
(a-1) providing a p-doped crystalline silicon substrate;
(a-2) etching pores into a surface of the p-doped crystalline silicon substrate by using an acid etchant solution and by applying an anodizing current, thereby forming a porous silicon layer in the p-doped crystalline silicon substrate, the porous silicon layer comprising the porous surface; and
(a-3) annealing the porous silicon layer.

14. The process of claim 12, wherein the p-doped silicon substrate comprises a dopant selected from the group consisting of boron, aluminum, gallium, indium, and combinations thereof.

15. The process of claim 12, wherein the conductive polymer is selected from the group consisting of polyanilines, polyphenylenes, polyphenylene vinylenes, polythiophenes, polypyrroles, polyfurans, polyselenophenes, polyisothianapthenes, polyphenylene sulfides, polyacetylenes, polydiacetylenes, polypyridyl vinylenes, polycarbazoles, conductive carbohydrates, conductive polysaccharides, derivatives thereof, blends thereof with other polymers, copolymers of the monomers thereof, and combinations thereof.

16. The process of claim 12, wherein the conductive polymer comprises one or more of an electrolyte-doped polypyrrole, polyaniline, and polythiophene.

17. The process of claim 12, wherein electrodepositing the lawn of the conductive polymer comprises electropolymerizing one or more conductive monomers by at least one of cyclic voltammetry, chronoamperometry, and chronopotentiometry.

18. The process of claim 12, wherein electrodepositing the lawn of the conductive polymer further comprises electrodepositing at least a portion of the conductive polymer beneath the porous surface and within pores of the p-doped silicon substrate.

19. The process of claim 12, wherein the binding pair member is selected from the group consisting of antibodies, antibody fragments, antigens, biotin, avidin and derivatives thereof, hormones, hormone receptors, polynucleotides, oligonucleotides, aptamers, whole cells, and combinations thereof.

20. The process of claim 12, wherein the binding pair member comprises a probe DNA (pDNA) oligonucleotide and part (c) comprises electrostatically binding the pDNA to the conductive polymer.
